(19) United States
(12) Patent Application Publication
(10) Pub. No.: US 2004/0014693 A1
(43) Pub. Date: Jan. 22, 2004

(54) Use of arginine in the preparation of a medicament for the prevention and treatment of the side effects associated with the intravenous administration of pharmaceuticals

(76) Inventors: Lorena Muggetti, Meda (IT); Alessandro Martini, Milan (IT); Giovanni Buzzi, Milan (IT); Paolo Colombo, Milan (IT)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/363,998

(22) PCT Filed: Sep. 7, 2001
(86) PCT No.: PCT/EP01/10398

(51) Int. Cl. A61K 31/704; A61K 31/198; A61K 31/337

(52) U.S. Cl. 514/34; 514/449; 514/565

(57) ABSTRACT

The present invention relates to the use of arginine and, more in particular, to the injectable formulations for intravenous use comprising it, in the prevention and treatment of the side effects associated with the extravasation of drugs administered by intravenous route.

The present invention relates to the use of arginine and, more in particular, of the injectable formulations thereof, in the prevention and treatment of the side effects associated with the intravenous administration of some pharmaceuticals.

It is known, in therapy, that several drugs administered through intravenous route may cause, at least potentially, vascular damages at the site of injection and ulcerative damages at the surrounding tissues, upon extravasation.

During an intravascular administration, extravasation is observed by fluid spillage, typically of blood, of a solution injected through intravenous route or of a mixture of both, from a blood or lymphatic vessel in the surrounding perivascular tissue.

The extravasation phenomenon may occur in several situations, for instance accidentally by means of a missed positioning of the needle inside the vein during the intravenous administration of the drug, with consequent leakage of the medicated solution into the surrounding perivascular tissue.

Alternatively, it is possible to get extravasation also in the case of a vein too small, for instance in relation to the administration speed and/or to the volume of injected solution, or already damaged by a previous administration of the drug or by a trauma.

The extravasation of some pharmaceuticals administered by intravenous route can lead to ulcerations particularly painful and spread which require, in some cases, the surgical removal of the interested tissue.

The extravasation phenomenon associated with the intravenous administration of drugs, particularly evident in the case of cytotoxic antineoplastic agents used in chemotherapy, is widely reported in the literature.

See, for instance, Proc. Annu. Meet. Am. Soc. Clin. Oncol. 13: A1627, 1994; Seminars in Oncology, 27(3):347-61, June 2000; Drug Safety, 12(4):245-255, April 1995; Nuritinga: An electronic journal of nursing ISSN 1440-1541 http://www.healthsci.utas.edu.au/nursing/nuritinga/vol2/sto ios.html).

Besides the above chemotherapeutic agents, either of synthetic or natural source, other classes of drugs administered by intravenous route may exert this kind of damaging action if, following extravasation, come in to contact with perivascular tissues.

We refer, for instance, to drugs having antibiotic, antifungine and also sedative activity.

More in general, compounds which may cause ulcerative damages following extravasation comprise antineoplastic agents such as, for instance, tubulin antagonists, alkylating agents, antibiotics, antimetabolites, topoisomerase inhibitors, angiogenesis inhibitors and platinum derivatives. In addition, the above side effects may also occur with other drugs such as, for instance, antiviral or vaso-suppressant agents and benzodiazepines.

In this respect, examples of specific compounds which may cause ulcerative damages by extravasation include, for instance, amsacrine, vincristine, vinblastine, vinorelbine, vindesine, gemcitabine, etoposide, dacarbazine, streptozocin, daunorubicin, idarubicin, epirubicin, doxorubicin, alkycyclin (4-demethoxy-3′-deamino-3′-aziridinyl-4′-methylsulfonyl-daunorubicin; internal code: PNU 159548), plicamycin, penicillin, vancomycin, chloramphenicol, bleomycin, mitomycin, actinomycin D, paclitaxel, docetaxel, Sugen SU-5416, Sugen SU-6668, amphotericin B, cisplatin, carboplatin, iphosphamide; fluorouracil, mechlorethamin, mustine, carmustine, estramustine, irinotecan, topotecan, epinephrine, norepinephrine, dopamine, dobutamine.

From a clinical point of view there are no consolidated therapies, and of certain efficacy, for the prevention and treatment of these lesions/ulcerations which, in the most serious cases and in the absence of suitable treatments, can progress up to necrosis of the interested tissues.

Some possible remedies are nevertheless known in the art to decrease the local toxicity of some of the most common drugs which show a damaging action following extravasation. See, for instance, the topical administration of dimethylsulphoxide in the case of anthracycline or mitomycin extravasation [Seminars in Oncology, 27(3):347-61, June 2000], the local injection of hyaluronidase in the case of alkaloids [Proc. Annu. Meet. Am. Chem. Soc. Clin. Oncol., 13:A1627, 1994) or the local injection of sodium thiosulphate in the case of mechlorethamine [Drug Safety, 12(4):245-255, April 1995].

As already reported, in the most unfavorable cases wherein the above remedies do not seem to exert the desired effect, the recurrence of plastic surgery remains the only possibility of intervention.

With the aim of minimize the unwanted effects associated with the intravenous administration of therapeutic agents able, at least potentially, to cause ulcerative damages by extravasation, specific formulations are known in the art. See, for instance, the liposomal anthracycline formulations with improved tolerability profile in comparison to the conventional formulations [Journal of Controlled Release, 53(1-3):275-9, Apr. 30, 1998] and the use of the cyclodextrins in the preparation of formulations for parenteral route (U.S. Pat. No. 5,804,568 in the name of Supergen Inc).

It is known, nevertheless, that the encapsulation in liposomes as well as the inclusion/association with cyclodextrins of various active principles may lead, in general, to a remarkable variation of the pharmacokinetic profile of the therapeutic substance itself.

Now we have unexpectedly found that arginine (The Merck Index, XII Ed. No. 817) results to be particularly effective in the prevention and treatment of the side effects due to the extravasation phenomena associated with the intravenous administration of some drugs.

The use of basic amino acids and, more particularly, of arginine, in the manufacture of intravenous formulations of estramustine is also reported in the international patent application WO 01/19372 in the name of the applicant itself.

As reported therein, however, arginine acts against possible thromophlebitis which are known to occur at the site of injection upon intravenous administrations of estramustine.

It is therefore an object of the present invention the use of arginine, and of the pharmaceutically acceptable salts thereof, in the preparation of a medicament for the prevention and treatment of the side effects associated with the extravasation of drugs administered by intravenous route.

According to a preferred embodiment of the invention, with the term arginine it is intended the essential amino acid in its optical active form L-arginine, optionally in the form of pharmaceutically acceptable salt for parenteral administration.

Pharmaceutically acceptable salts comprise the acid addition salts with organic or inorganic acids such as, for instance, hydrochloric, glutamic and aspartic acid.

Preferably, the subject invention relates to the use of arginine or arginine hydrochloride.

For the safety and tolerability profile which characterize arginine and its pharmaceutically acceptable salts, for its easy availability and versatility of use, the subject invention results to be particularly advantageous, in therapy, in the intravenous administration of several drugs.

According to a preferred embodiment of the invention, arginine results to be particularly advantageous in the prevention and treatment of perivascular damages associated with the intravenous administration of drugs with antitumor activity such as, for instance, tubulin antagonists, alkylating agents, antibiotics, antivirals, antimetabolites, topoisomerase inhibitors, angiogenesis inhibitors and platinum derivatives.

Particularly preferred, in this respect, is the use of arginine in antitumor therapy comprising the intravenous administration of anthracyclines and derivatives such as doxorubicin, epirubicin, idarubicin, daunorubicin, alkycyclin (4-demethoxy-3′-deamino-3′-aziridinyl-4′-methylsulfonyl-daunorubicin; internal code: PNU 159548), taxanes such as paclitaxel and docetaxel; estramustine phosphate; Sugen SU-5416 and Sugen SU-6668; either used as single agents or in association with other conventional chemotherapeutic agents.

Arginine, according to the subject of the present invention, can be administered either contemporaneously or sequentially to the administration of the drug to be injected by intravenous route.

In the first case, the arginine may be present as a constituent of the formulation itself.

As an example, according to the type of drug to be employed, arginine may be present either in combination with the active principle, in the form of arginine salt, or as additional ingredient, together with any other pharmaceutical excipients for parenteral use.

A typical example of formulation able to prevent the ulcerative phenomena following the possible extravasation of estramustine phosphate, when administered by intravenous route, is just a formulation comprising estramustine phosphate as the arginine salt, as reported in the examples.

Alternatively, as above indicated, arginine may be present in the solution containing the active principle to be intravenously injected, as an additional ingredient.

In such a case, for instance in the therapy comprising the intravenous administration of estramustine phosphate, the active principle may be constituted by a pharmaceutically acceptable salt, for instance the salt with N-methyl-glucamine, otherwise known as meglumine.

In addition to what above indicated and for the safety profile which characterize arginine, the same may be present as a salt in the formulation to be injected, in combination with the active principle, and also as additional ingredient.

In preparing such a formulation, it is clear to the skilled man that more than one equivalent of arginine per equivalent of active principle, are needed.

From all of the above, analogous considerations may apply to formulations for intravenous use comprising an active principle other than estramustine phosphate and in any case capable of causing ulcerative phenomena following extravasation.

As above indicated, arginine may be also administered separately to the active principle, for instance by working as described in the literature for solutions containing thiosulfate or hyaluronic acid to be locally used once extravasation phenomena are observed.

In such a case, a physiological injectable arginine solution, optionally in association with other pharmaceutically acceptable excipients, may be administered through local injection in the proximity of the area damaged by the previous intravenous administration of the drug.

Among the pre-clinical studies which are needed for a drug to be administered intravenously, there is the evaluation of any local reaction in case of partial accidental administration of the drug outside the vessel itself.

Such a study, for instance carried out according to the experimental model described below, allows to evaluate the irritant/histological-damaging capability of drugs administered by intravenous route, once extravasated. Generally, even if the clinical and histological examination of the site of injection in the repeated toxicological studies allows to give an indication of the local tolerability of a drug, it is anyway preferred to carry out an ad hoc study.

In this respect, any result obtained in the animal model is useful to understand whether a possible accidental extravasation, in the clinical use, may lead to the aforementioned inconvenients at the site of administration. With the aim of mimic a possible clinical situation, the model which is usually considered is the paravenous administration (marginal vein) at the rabbit ear.

As an example, a limited amount of the compound to be tested (0.3-0.5 ml) is injected at the peri-vasal site; the inoculation site is carefully examined for at least one week.

To evaluate possible alterations at the site of injection in the most correct and possible objective manner, it is used a “score” system.

Attention is mainly given to the presence of erythema, inflammatory edema and the possible appearances of eschar, ulcerations or crust lesions. Generally, each animal is the control of itself and the opposite ear receives the vehicle, that is the same solution not containing the active principle.

The most elevated concentration of the compound to be tested is the maximum concentration intended for clinical practice.

Usually, two animals are sacrificed: one in the acute phase, after the administration of the drug (48-72 hours) and another later, at least a week after the administration of the drug.

It is thus carried out the histological examination of all the sites of injection.

As above reported, arginine may be present in the formulation to be injected to prevent and treat the damages of extravasation, either in combination and/or association with one or more active principle or, alternatively, per se plus conventional physiological excipients.

Said formulations are prepared according to conventional techniques used in the preparation of pharmaceutical forms for intravenous administration and may also contain other pharmaceutically acceptable excipients for parenteral use such as, for instance bulking agents (e.g. lactose or mannitol), pH buffering, antioxidants, preservatives, tonicity adjusters and the like.

The following examples are intended to better illustrate the present invention without posing any limitation to it.

### EXAMPLE 1

Preparation of the Salt of Estramustine Phosphate with Arginine

300 mg of estramustine phosphate were weighed in a beaker and dispersed in 5 ml of water under magnetic stirring. 101 mg of arginine base were then added, under stirring, to the aqueous dispersion of the active principle and, after few minutes, a clear solution was obtained.

The solution thus prepared was then diluted with water up to a final volume of 10 ml so as to reach a final concentration of 30 mg/ml of estramustine phosphate and 10.1 mg/ml of arginine (molar ratio 1:1, respectively).

A solution prepared as above described, properly sterilized by filtration, was tested for its local tolerability in the animal, following extravasation.

### EXAMPLE 2

Preparation of the Salt of Estramustine Phosphate with Arginine, in Admixture with Arginine

300 mg of estramustine phosphate were weighed in a beaker and dispersed in 5 ml of water under magnetic stirring. 202 mg of arginine base were then added, under stirring, to the aqueous dispersion of the active principle and, after few minutes, a clear solution was obtained. The basic pH of the obtained solution was brought to the physiological value of about 7.5 by slow addition of hydrochloric acid.

The solution thus prepared was then diluted with water up to a final volume of 10 ml so as to reach a final concentration of 30 mg/ml of estramustine phosphate and 20.2 mg/ml of arginine (molar ratio 1:2, respectively).

A solution prepared as above described, properly sterilized by filtration, was tested for its local tolerability in the animal following extravasation.

### EXAMPLE 3

Preparation of the Salt of Estramustine Phosphate with N-Methyl-Glucamine, in Admixture with Arginine

300 mg of estramustine phosphate were weighed in a beaker and dispersed in 5 ml of water under magnetic stirring. 120.8 mg of N-methyl-glucamine were then added, under stirring, to the aqueous dispersion of the active principle and, after few minutes, a clear solution was obtained. To the prepared solution was then added, under stirring, an amount of arginine corresponding to 202 mg by using a proper admixture of arginine base and arginine hydrochloride so as to maintain the final pH as closer as possible to the physiological pH (about 7.5). The solution thus prepared was then diluted with water up to a final volume of 10 ml so as to reach a final concentration of 30 mg/ml of estramustine phosphate and 20.2 mg/ml of arginine (molar ratio 1:2, respectively).

A solution prepared as above described, properly sterilized by filtration, was tested for its local tolerability in the animal following extravasation.

### EXAMPLE 4

The formulation described in the previous example was also prepared by dissolving the lyophilized formulation of commercially available Estracyt® containing 300 mg per vial of the active principle.

The reconstitution of the formulation was carried out by using 10 ml of a solution containing 20.2 mg/ml of arginine so as to reach a final concentration of 30 mg/ml of estramustine phosphate and 20.2 mg/ml of arginine (molar ratio 1:2, respectively).

The solution of arginine used to dissolve the commercial lyophile was prepared by dissolving in water proper amounts of arginine base and hydrochloride so as to obtain a final concentration of 20.2 mg/ml and a pH value as closer as possible to the physiological one (about 7.5).

### EXAMPLE 5

Preparation of a Formulation Containing Doxorubicin and Arginine in a Molar Ratio 1:1

40 mg of doxorubicin hydrochloride and 12 mg of arginine were weighed in a 20 ml flask. The admixture was then dissolved in 15 ml of physiological solution (NaCl 0.9% w/v) under magnetic stirring. To the solution thus obtained a solution of HCl up to pH=3 was then added. The solution thus prepared was then diluted with the above physiological solution up to a final volume of 20 ml so as to reach a final concentration of 2 mg/ml of doxorubicin and 0.6 mg/ml of arginine (molar ratio 1:1, respectively).

A solution prepared as previously described, properly sterilized by filtration, was tested for its local tolerability in the animal following extravasation.

### EXAMPLE 6

Preparation of a Formulation Containing Doxorubicin and Arginine in a Molar Ratio 1:2

By working analogously to what described in example 5 and by using an amount of arginine as twice, that is 24 mg of arginine per 40 mg of doxorubicin hydrochloride, it was prepared a solution containing doxorubicin and arginine in a molar ratio 1:2, respectively.

A solution prepared as previously described, properly sterilized by filtration, was tested for its local tolerability in the animal following extravasation.

### EXAMPLE 7

Preparation of a Formulation for Intravenous Use Containing Sugen SU 5416 and Arginine in a Molar Ratio 1:1

In a graduated flask of 20 ml there were diluted 10 ml of an aqueous solution of NaCl (0.9% w/v) with 10 ml of water with the aim of obtaining a solution at 0.45% w/v of sodium chloride.

39.79 mg of arginine hydrochloride were then dissolved into the solution thus obtained by simple manual shaking.

The solvent solution thus prepared was then used to dilute a solution of the compound Sugen SU 5416 having the following composition:

| Component | Amount % (w/v) within the formulation |
|---|---|
| Sugen SU 5416 | 0.45% |
| PEG 400 | 45% |
| Benzyl alcohol | 2% |
| Cremophor EL | 31.5% |
| Anhydrous ethanol | q.b. to 1000 |

The dilution was carried out by mixing a part of the formulation containing the active principle with two parts of the solvent containing arginine so as to obtain a solution containing Sugen SU 5416 and arginine in a molar ratio 1:1.

A solution prepared as previously described, properly sterilized by filtration, was tested for its local tolerability in the animal following extravasation.

### EXAMPLE 8

Preparation of a Formulation for Intravenous Route Containing Sugen SU 5416 and Arginine in a Molar Ratio 1:2

By working analogously to what reported in example 7 but using 79.58 mg of arginine hydrochloride, it was obtained a solution containing Sugen SU 5416 and arginine in a molar ratio 1:2, respectively.

A solution prepared as previously described, properly sterilized by filtration, was tested for its local tolerability in the animal following extravasation.

1. Use of arginine, and of its pharmaceutically acceptable salts, in the preparation of a medicament for the prevention and treatment of the side effects associated with the extravasation of drugs administered by intravenous route.

2. Use according to claim 1 wherein the arginine is in the form of arginine base or as hydrochloride salt.

3. Use according to claim 1 wherein the drugs administered by intravenous route are antineoplastic agents.

4. Use according to claim 3 wherein the antineoplastic agents are selected from tubulin antagonists, alkylating agents, antibiotics, antivirals, antimetabolites, topoisomerase inhibitors, angiogenesis inhibitors and platinum derivatives, either used alone or in combination or in association with other conventional chemotherapeutic agents.

5. Use according to claim 3 wherein the antineoplastic agent is selected from doxorubicin, epirubicin, idarubicin, daunorubicin, alkycyclin (4-demethoxy-3′-deamino-3′-aziridinyl-4′-methylsulfonyl-daunorubicin; internal code: PNU 159548), paclitaxel, docetaxel, estramustine phosphate, Sugen su-5416 and Sugen SU-6668, and pharmaceutically acceptable salts thereof; either used as single agents or in combination or in association with other conventional chemotherapeutic agents.

6. Use according to claim 5 wherein the antineoplastic agent is estramustine phosphate, doxorubicin or Sugen SU 5416, and pharmaceutically acceptable salts thereof.

7. Use of a composition for intravenous administration comprising an antineoplastic agent and arginine, in the preparation of an antitumor medicament which prevents and treats the side effects associated with the extravasation of the said antineoplastic agent.

8. Use according to claim 7 wherein the antineoplastic agent is selected from doxorubicin, Sugen SU 5416, estramustine phosphate and pharmaceutically acceptable salts thereof.

9. Use according to claim 7 wherein arginine is in the form of arginine base or as pharmaceutically acceptable salt thereof.

10. Use according to claim 7 wherein the antineoplastic agent is in the form of arginine salt.

11. A product or kit for use in antitumor therapy comprising:
i) an intravenous formulation of an antineoplastic agent; and
ii) a parenteral formulation of arginine or a pharmaceutically acceptable salt thereof;
for the prevention and treatment the side effects associated with the extravasation of the above antineoplastic agent.

12. A method for preventing and treating the side effects associated with the extravasation of an antineoplastic agent administered by intravenous route which comprises administering to a mammal in need thereof the said antineoplastic agent and arginine.

13. The method of claim 12 wherein arginine is in the form of arginine base or as pharmaceutically acceptable salt thereof.

14. The method of claim 12 wherein the antineoplastic agent is selected from tubulin antagonist, alkylating agents, antibiotics, antivirals, antimetabolites, topoisomerase inhibitors, angiogenesis inhibitors and platinum derivatives, either used alone or in combination or in association with other conventional chemotherapeutic agents.

15. The method of claim 14 wherein the antineoplastic agent is selected from doxorubicin, epirubicin, idarubicin, daunorubicin, alkycyclin (4-demethoxy-3′-deamino-3′-aziridinyl-4′-methylsulfonyl-daunorubicin; internal code: PNU 159548), paclitaxel, docetaxel, estramustine phosphate, Sugen SU-5416 and Sugen SU-6668, and pharmaceutically acceptable salts thereof; either used as single agents or in combination or in association with other conventional chemotherapeutic agents.

16. The method of claim 15 wherein the antineoplastic agent is estramustine phosphate, doxorubicin or Sugen SU 5416, and pharmaceutically acceptable salts thereof.

17. The method of claim 12 wherein the antineoplastic agent is in the form of arginine salt.

18. The method of claim 12 wherein the mammal in need thereof is a human.

19. A formulation for intravenous use comprising arginine or a pharmaceutically acceptable salt thereof, and an antineoplastic agent selected from doxorubicin, epirubicin, idarubicin, daunorubicin, alkycyclin (4-demethoxy-3′-deamino-3′-aziridinyl-4′-methylsulfonyl-daunorubicin; internal code: PNU 159548), paclitaxel, docetaxel, Sugen SU-5416 and Sugen SU-6668, and pharmaceutically acceptable salts thereof; either used as single agents or in combination or in association with other conventional chemotherapeutic agents.
